(19) United States
(12) Patent Application Publication
(10) Pub. No.: US 2025/0230433 A1
(43) Pub. Date: Jul. 17, 2025

(54) PHAGE CHARACTERIZATION METHOD AND DEVICES

(71) Applicant: HOGESCHOOL GENT, Gent (BE)

(72) Inventor: Stefan VERMEULEN, Parike (BE)

(21) Appl. No.: 18/702,870

(22) PCT Filed: Oct. 20, 2022
(86) PCT No.: PCT/EP2022/079181

§ 371 Date: Apr. 19, 2024

(30) Foreign Application Priority Data
Oct. 20, 2021 (EP) 21203671.9

(51) Int. Cl. C12N 15/10; C12M 1/00; C12M 1/26; C12M 1/34; C12M 3/06; C12Q 1/02

(52) U.S. Cl. CPC C12N 15/1082; C12M 23/16; C12M 29/06; C12M 33/04; C12M 41/46; C12Q 1/025; G01N 2333/005

(57) ABSTRACT

Disclosed herein is a method for characterizing a phage solution in a microfluidic device comprising the steps of mixing a phage solution or a dilution thereof with a bacterial solution, thereby generating a phage/bacteria solution; and through a set of microfluidic channels of said microfluidics device pumping the phage/bacteria solution to a droplet generator unit, thereby generating phage/bacteria droplets; incubating the generated phage/bacteria droplets in a transparent incubation chamber; and; monitoring, in function of time, the characteristics of the phage/bacteria droplets in said incubation chamber.

### FIELD OF THE INVENTION

The present invention relates to a method for characterizing a phage solution in a microfluidic device.

### BACKGROUND

The recent introduction of droplet microfluidic technologies to studies in microbiology has opened new possibilities in detection and identification of pathogens, antibiotic susceptibility testing, studies of microbial physiology and biotechnological applications. These technologies are typically based on the encapsulation of microorganisms in droplets and the use of these droplets to study these microorganisms and allow the use of small volumes, the ability to work with very large numbers of droplet reactors, and the capability to incorporate complex liquid handling protocols in large numbers of droplets.

However, for phage-based detection assays droplet microfluidic technologies have not yet been fully developed. Whereas droplet microfluidic technologies for phage detection have been developed for clinical diagnostics, the monitoring of water quality, and food safety, these applications merely determine the presence or absence of certain phages. In order to characterize the phages, researchers often have to return to more conservative techniques where phages are visualized as plaques in a lawn of bacteria on an agar-filled Petri dish allowing the phages to be further characterized. However typically the conservative techniques require a lot of manual handling and up to 12 h of incubation time and the development of a signal from for instance a reporter protein could also require lengthy time for development of a detectable and quantifiable signal. Also, PCR-based techniques require complicated labelling techniques, time-consuming culture assays and suffer low recovery rate. Also, PCR based techniques focusses on DNA strands and does not distinguish between live or dead bacteriophages. Thus, there exists a need for methods, products and tools that would improve the characterization of phage solutions. Such methods, products and tools are described herein.

### SUMMARY

Accordingly, in a first aspect, the present invention provides in a method for characterizing a phage solution in a microfluidic device comprising the steps of:
mixing a phage solution or a dilution thereof with a bacterial solution, thereby generating a phage/bacteria solution; and through a set of microfluidic channels of said microfluidics device:
pumping the phage/bacteria solution to a droplet generator unit, thereby generating phage/bacteria droplets;
incubating the generated phage/bacteria droplets in a transparent incubation chamber; and;
monitoring, in function of time, the characteristics of the phage/bacteria droplets in said incubation chamber.

More in particular, the method as disclosed herein provides that the monitoring of the characteristics of the phage/bacteria droplets comprises at least one of:
a quantitative assessment of the bacterial solution which includes determining the concentration of bacteria in the bacterial solution, preferably by determining the turbidity of the bacterial solution, the optical density of the bacterial solution and/or the colony forming units per ml (cfu/ml);
a qualitative assessment of the bacterial solution which includes the fitness, viability and/or identification of the bacterial solution;
a quantitative assessment of the phage solution which includes determining the concentration of viable phages in the phage solution, preferably by determining the plaque forming units per ml (pfu/ml); and/or
a qualitative assessment of the phage solution which includes the fitness and/or specificity of the phage solution.

Furthermore, the method as disclosed herein provides that the step of mixing a phage solution or a dilution thereof with a bacterial solution occurs on the microfluidic device.

More in particular, the method as disclosed herein provides that the incubation step occurs under predetermined conditions which include a predetermined incubation temperature, a predetermined atmosphere and a predetermined incubation time.

Furthermore, the method as disclosed herein provides that the step of monitoring, in function of time, the characteristics of the phage/bacteria droplets in said incubation chamber includes obtaining a control image at the start of the incubation period and obtaining a final image at the end of the incubation period, and optionally obtaining images at preset intervals during the incubation period. In a particular embodiment, the method as disclosed herein provides that between 1000 and 25000 phage/bacteria droplets are simultaneously monitored in said transparent incubation chamber.

More in particular, the method as disclosed herein provides that the method further comprises the step of guiding incubated phage/bacteria droplets to a droplet selector and collecting selected phage/bacteria droplets.

In a further aspect, disclosed herein is a microfluidic phage solution characterization device, comprising:
a fluid input;
a fluid output;
one or more microfluidic channels fluidically connecting the fluid input to the fluid output wherein each microfluidic channel comprises a droplet generator unit for generating phage/bacteria droplets, a transparent incubation chamber for incubating the generated phage/bacteria droplets and a monitoring system for, in function of time, determining the characteristics of phage/bacteria droplets in said incubation chamber.

More in particular, the microfluidic device as disclosed herein is characterized by comprising:
a first fluid input for a phage solution;
a second fluid input for a bacterial solution;
a third fluid input for an oily solution;
optionally a fourth fluid input for a titration solution;

and wherein each microfluidic channel further comprises a mixer unit for mixing the phage solution or a dilution thereof with a bacterial solution, thereby generating a phage/bacteria solution.

Furthermore, the microfluidic device as disclosed herein provides that the droplet generator unit is a passive or active droplet generator unit.

In an additional embodiment, the microfluidic device as disclosed herein provides that the transparent incubation chamber has a height between 50 μm and 500 μm.

More in particular, the microfluidic device as disclosed herein provides that the transparent incubation chamber has a flat or waved surface.

Furthermore, the microfluidic device as disclosed herein provides that the microfluidic device further comprises at least one of:
a heating element for controlling the temperature of one or more incubation chambers;
a droplet mixer unit associated with each microfluidic channel for mixing each generated phage/bacteria droplet;
an inertial pump associated with each microfluidic channel, each inertial pump including a fluid actuator to pump fluid in/out the microfluidic channel;
a droplet selector unit associated with each microfluidic channel;
a UV illuminator;
an atmospheric control unit for controlling the atmospheric conditions during incubation;
a recirculation conduit for recirculating specific droplets to the input of the phage solution;
a phage selection/isolation unit for isolating and/or selecting a phage sample from a phage bank, an environmental sample;
an upscaling unit for preparing a phage starter sample from a selected droplet;
an upscaling unit for preparing a bacterial starter sample from a selected droplet;
a phage solution titration unit; and/or;
a bacterial solution titration unit.

In a further aspect, disclosed herein is a microfluidics-based platform comprising the microfluidic device as disclosed herein and an analysis system for processing the data obtained from the monitoring system.

In a further aspect, disclosed herein is the use of the microfluidic device as disclosed herein for characterizing a phage solution comprises at least one of:
a quantitative assessment of the phage solution which includes determining the concentration of viable phages in the phage solution, preferably by determining the plaque forming units per ml (pfu/ml); and/or
a qualitative assessment of the phage solution which includes the fitness and/or specificity of the phage solution.

### FIGURES

FIG. 1 illustrates a device as disclosed herein.

FIG. 2: Representative images of approximately 12 000 S aureus-ISP droplets analysed with two consecutive methods. A suspension of 10^{E}8 CFU/ml bacteria were challenged with 10^{E}6 PFU/ml ISP phages. The upper panels show a part of an unprocessed grey value image (400×500 pixels) at several time intervals 2 h, 4 h, 6 h and 24 h. The middle panel shows histograms of the relative mean intensity of the individual droplets of the full image after image processing and parameter analysis. The horizontal axis represents the relative mean intensity and the amount of droplets is represented in the vertical axis. The lower panel demonstrates a statistical analysis of the full images where the processed droplets were randomized. The horizontal axis represents the randomized droplets, the vertical axis represents the baselined and inverted mean grey values. The arrows in the column of 24 h represents in the upper panel a droplet where bacteria have been lysed by phages producing a clear droplet and in the middle and lower panel the droplet population which had received the phages and which were lysed.

FIG. 3: Representative visualizations of 12 000 S aureus (10⁸ CFU/ml) droplet cultures challenged with different concentrations (10⁶, 10⁷ 10⁸ PFU/ml) of ISP phages in function of time.

FIG. 4: Representative comparative study of DAO method and microfluidic method. 3 independent manual titrations were done. The horizontal axis represents the concentration obtained by the DAO method. The vertical axis represents the concentration obtained by the microfluidic method. Two separate samples with known titer were compared. Note the linearity over a broad range of concentrations. Stdev for the microfluidic method over all measurements ranged from 0.05 to 0.0004 and are, hence, not clearly visible in the plot.

FIG. 5 illustrates a device as disclosed herein.

FIG. 6A and 6B respectively show a detail of the droplet generator unit (3) and the incubation chamber (4).

FIG. 7 illustrates a device as disclosed herein.

### DETAILED DESCRIPTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

All references cited in this description are hereby deemed to be incorporated in their entirety by way of reference.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, in particular +/−10% or less, more in particular +/−5% or less, even more in particular +/−1% or less, and still more in particular +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “weight percent”, “% wt” or “weight %”, here and throughout the description unless otherwise defined, refers to the relative weight of the respective component based on the overall weight of the formulation.

Disclosed herein are methods and devices that are based on microfluidic droplet technologies and enable an assessment of bacteriophages which can for instance be used for phage therapy or phage monitoring in relevant biological systems. The methods and devices as disclosed herein are for instance able to determine the quantity, fitness and specificity of the bacteriophages for a specific host bacteria. On the basis of this information it is possible to determine correct phage therapy dose administration (in pfu/ml) to a patient, animal or crop. The methods and devices as disclosed herein can also be used to investigate sources of contamination in bioplants, to correctly quantify phages in a production facility or for selecting specific phages suitable for specific species or strains of bacteria.

Accordingly, in a first aspect, the present invention provides in a method for characterizing a phage solution in a microfluidic device comprising the steps of:
mixing a phage solution or a dilution thereof with a bacterial solution, thereby generating a phage/bacteria solution; and through a set of microfluidic channels of said microfluidics device:
pumping the phage/bacteria solution to a droplet generator unit, thereby generating phage/bacteria droplets;
incubating the generated phage/bacteria droplets in a transparent incubation chamber; and;
monitoring, in function of time, the characteristics of the phage/bacteria droplets in said incubation chamber.

As referred to herein the term “microfluidic device” or “microfluidic chip” refers to a device that enables a tiny amount of liquid to be processed or visualized. The dimension of the chip typically ranges between 1 cm and 10 cm, with a chip thickness ranging from about 0.5 mm to 5 mm. Microfluidic devices/chips are characterized by comprising internal hair-thin microfluidic channels that are connected to the outside by means of holes on the chip referred to as inlet/outlet ports. Microfluidic chips are typically made from thermoplastics such as acrylic, glass, silicon, PDMS (polydimethylsiloxane), PMMA (polymethylmetacrylate), COC (cyclo-olefin-copolymer), COP (cyclo-olefinpolymer), PC (polycarbonate) or PS (polystyrene). Typically microfluidic systems transport, mix, separate, or otherwise process fluids. Also as used herein the term “droplet-based microfluidics” refers to a subcategory of microfluidics which in contrast with continuous microfluidics manipulates discrete volumes of fluids in immiscible phases with low Reynolds number and laminar flow regimes. Interest in droplet-based microfluidics systems has been growing substantially in past decades. Microdroplets allow for handling miniature volumes (μl to fl) of fluids conveniently, provide better mixing, encapsulation, sorting, and sensing, and suit high throughput experiments. However, exploiting the benefits of droplet-based microfluidics efficiently requires a deep understanding of droplet generation and depends on the types of liquid solutions to which droplet-based microfluidics are applied.

By using microfluidic devices the methods and devices as disclosed herein allow for a fast assessment of the phage solution and/or the bacterial solution. Also, it provides for methods that are more economical as less consumables are used and also less waist is generated. Finally the use of microfluidic devices allows for a more automated process in the handling of samples, the incubation and the actual read-out of the results.

As referred to herein the terms “phage solution” and “bacterial solution” refer to liquid solutions comprising respectively phages and bacteria that are used in the methods and devices disclosed herein. As referred to herein the term “droplet generator unit” refers to a unit integrated on the microfluidic devices that generates droplets of the phage/bacteria solution. The droplet generator unit may use active or passive techniques for the generation of the droplets. Passive droplet generation techniques are typically based on microfluidic structures that provide in the droplet formation by for instance co-flow, cross-flow and flow-focusing. Preferably the cross-flow microfluidic structures used to generate droplets in the microfluidic device as disclosed herein are preferably chosen from a T-junction, a double T-junction, a Y-junction, a head-on junction or an alternating T-junction. Preferably the co-flow and/or flow-focusing microfluidic structures used to generate droplets in the microfluidic device as disclosed herein are preferably chosen from an orifice (in flow-focusing or co-flow) or emulsification methods. Active droplet generation techniques are typically based on active external actuations including electrical, magnetic, thermal, acoustic and mechanical methods. In preferred embodiments droplet generator unit as disclosed herein uses pneumatic or piezoelectric actuation to generate droplets.

As referred to herein the term “transparent incubation chamber” refers to the enclosed space on the microfluidic chip as disclosed herein where the generated droplets of the phage/bacteria solution are maintained and monitored for a certain period. To enable adequate monitoring of the droplets the incubation chamber is made transparent such that the monitoring may occur through various techniques including spectral imaging.

In a particular embodiment the method as disclosed herein provides that the monitoring of the characteristics of the phage/bacteria droplets comprises at least one of:
a quantitative assessment of the bacterial solution which includes determining the concentration of bacteria in the bacterial solution, preferably by determining the turbidity of the bacterial solution, the optical density of the bacterial solution and/or the colony forming units per ml (cfu/ml);
a qualitative assessment of the bacterial solution which includes the fitness, viability and/or identification of the bacterial solution;
a quantitative assessment of the phage solution which includes determining the concentration of viable phages in the phage solution, preferably by determining the plaque forming units per ml (pfu/ml); and/or
a qualitative assessment of the phage solution which includes the fitness and/or specificity of the phage solution.

In particular, the quantitative assessment of the bacterial solution includes determining the concentration of bacteria in the bacterial solution, preferably by determining the turbidity of the bacterial solution, the optical density of the bacterial solution and/or the colony forming units per ml (cfu/ml). In particular, the quantitative assessment of the bacterial solution includes a measurement of the bacterial growth and the decline thereof (because of the phages multiplying and killing the bacteria).

In particular, the qualitative assessment of the bacterial solution includes the fitness, viability and/or identification of the bacterial solution. As referred to herein the term “fitness” refers to the ability of the bacteria to adjust their metabolism to suit environmental conditions. As referred to herein the term “viability” refers to the ability of the bacterial solution to grow and divide giving insights in the survival of the bacterial solution and the growth stage of the bacterial solution (e.g. lag phase, the log phase, the stationary phase, and the death phase). As referred to herein the term “identity” refers to species identification of the types of bacteria present in the bacterial solution.

In particular, the quantitative assessment of the phage solution includes determining the concentration of viable phages in the phage solution, preferably by determining the plaque forming units per ml (pfu/ml). In particular, the quantitative assessment of the phage solution includes a measurement of the phage multiplication and lysis in the solution.

In particular, the qualitative assessment of the phage solution includes the fitness and/or specificity of the phage solution. As referred to herein the term “fitness” refers to how fast does the phage lyses the bacteria. As referred to herein the term “specificity” refers to the phage lysing one or more specific family/genus/species/strain of bacteria.

The method as disclosed herein allows to provide in a label-free method that allows the simultaneous monitoring of more than 1000 individual phage/bacteria environments. Because the incubation occurs in a single incubation chamber the incubation occurs in a consistent manner with little to no room for deviations.

In a particular embodiment the method as disclosed herein provides that the step of mixing a phage solution or a dilution thereof with a bacterial solution occurs on the microfluidic device. While it is possible that the method as disclosed herein provides that a phage/bacteria solution is loaded onto a microfluidic device for further processing, in a preferred embodiment the method integrates all processing steps onto the microfluidic device. Accordingly, in a preferred embodiment the method includes the steps of loading both the phage solution and the bacterial solution separately in an inlet of the microfluidic device after which all steps of mixing, droplet formation, incubation and monitoring occur on the microfluidic device.

By providing as much operations as possible onto the microfluidic device, manual handling is limited and the reliability and repeatability of the methods are improved.

In a further embodiment, the method as disclosed herein provides that the incubation step occurs under predetermined conditions which include a predetermined incubation temperature, a predetermined atmosphere and a predetermined incubation time.

The method as disclosed herein therefore provides in a consistent and coherent incubation over time. The transparent incubation chamber further allows flexibility as to time intervals that are required during the monitoring as compared to devices where the imaging occurs on a droplet-by-droplet basis where repeated measurements of the same droplet over a certain time period is not possible.

In a particular embodiment the method as disclosed herein provides that the step of monitoring, in function of time, the characteristics of the phage/bacteria droplets in said incubation chamber includes obtaining a control image at the start of the incubation period and obtaining a final image at the end of the incubation period, and optionally obtaining images at preset intervals during the incubation period. This allows improved quantitative and qualitative assessments of the phages and bacteria over time, thereby not only providing a single data point with a positive or negative outcome (often requiring the use of detectable labels) but providing a time series showing the progress over time of the reaction occurring in each droplet.

In a particular embodiment the method as disclosed herein provides that between 1000 and 25000 phage/bacteria droplets are simultaneously monitored in said transparent incubation chamber. More in particular between 1000 and 25000 phage/bacteria droplets are simultaneously monitored in said transparent incubation chamber. It should be noted that as disclosed herein, a multitude of incubation chambers can be monitored at the same time.

In a further embodiment the method as disclosed herein provides that it comprises the step of guiding incubated phage/bacteria droplets to a droplet selector and collecting selected phage/bacteria droplets. By including onto the microfluidic device a droplet selector and collector it would allow selecting and collecting particular droplets which may be considered of interest to the user.

In a further aspect, disclosed herein is a microfluidic phage solution characterization device, preferably a device for performing the method as disclosed herein, comprising:
a fluid input;
a fluid output;
one or more microfluidic channels fluidically connecting the fluid input to the fluid output wherein each microfluidic channel comprises a droplet generator unit for generating phage/bacteria droplets, a transparent incubation chamber for incubating the generated phage/bacteria droplets and a monitoring system for, in function of time, determining the characteristics of phage/bacteria droplets in said incubation chamber.

As referred to herein the “monitoring system” refers to a measurement system for monitoring in function of time the phage/bacteria droplets in the incubation chamber. The monitoring system may be an off-chip or on-chip system and preferably provides in a universal imaging technique chosen from whole light spectrum monitoring or specific bandwidth monitoring.

In a particular embodiment the microfluidic phage solution characterization device as disclosed herein provides that it comprises 1 to 256 microfluidic channels fluidically connecting the fluid input to the fluid output wherein each microfluidic channel comprises a droplet generator unit for generating phage/bacteria droplets, a transparent incubation chamber for incubating the generated phage/bacteria droplets and a monitoring system for, in function of time, determining the characteristics of phage/bacteria droplets in said incubation chamber. In a particular embodiment the microfluidic phage solution characterization device as disclosed herein provides that it comprises 2 to 128, more preferably 4 to 64, and most preferably 8, 16 or 32 microfluidic channels fluidically connecting the fluid input to the fluid output wherein each microfluidic channel comprises a droplet generator unit for generating phage/bacteria droplets, a transparent incubation chamber for incubating the generated phage/bacteria droplets and a monitoring system for, in function of time, determining the characteristics of phage/bacteria droplets in said incubation chamber.

By providing a multitude of such microfluidic channels onto a single microfluidic device it would be possible to conduct a multitude of assessments on the phage and bacteria solutions, for instance including titrations of the solutions. In particular embodiments the titration of the input solutions occurs on the microfluidic device and each of the titrations are separately treated and incubated on the same microfluidic device, thereby conducting the experiments and generating the results of the different titrations in parallel. For instance, a single microfluidic device would be able to conduct a multiplicate of experiments, for instance providing a single device triplicate dilution series of 10⁵, 10⁷, 10⁹ and 10¹¹ to accurately perform a quantitative assessment of the phage or bacterial solutions.

Accordingly, in a particular embodiment, the microfluidic device as disclosed herein further comprises:
a first fluid input for a phage solution;
a second fluid input for a bacterial solution;
a third fluid input for an oily solution;
optionally a fourth fluid input for a titration and/or an antibacterial solution;
   and wherein each microfluidic channel further comprises a mixer unit for mixing the phage solution or a dilution thereof with a bacterial solution, thereby generating a phage/bacteria solution.

By providing the microfluidic channels with mixer units the phage/bacteria solution is uniformly mixed and the resulting droplets provide in a high contact ratio between the phages and hosts/bacteria thereby providing an improved efficiency of the incubation. Optionally the microfluidic channel further comprises a filter unit to remove debris and air bubbles from the system. The filter unit prevents clogging of the microfluidic system.

In a further embodiment, the microfluidic device as disclosed herein provides that the droplet generator unit is a passive or active droplet generator unit. More in particular, the passive droplet generator unit is preferably selected from T-junction, a double T-junction, a Y-junction, a head-on junction, an alternating T-junction, an orifice (in flow-focusing or co-flow) or an emulsification method. Alternatively, the active droplet generator unit is preferably selected from external actuations including electrical, magnetic, thermal, acoustic, mechanical, pneumatic or piezoelectric methods.

In a particular embodiment, the microfluidic device as disclosed herein provides that the transparent incubation chamber has a height between 50 μm and 500 μm. Providing an incubation chamber as disclosed herein allows for the incubation chamber to comprise an amount of droplets which provides a wide dynamic range. In particular embodiments the surface area of the incubation chamber ranges between 0.5 cm² and 4.0 cm², more in particular between 0.5 cm² and 2.0 cm², more in particular about 1 or 2 cm².

In a particular embodiment, the microfluidic device as disclosed herein provides that the transparent incubation chamber has a flat or waved surface. In a particular embodiment, the incubation chamber provides in a flat surface, thereby allowing a maximal capacity of droplets in the incubation chamber. Alternatively, in a particular embodiment, the incubation chamber provides in a waved surface, thereby allowing placement of the droplets on a fixed position in the incubation chamber, improving the measurement of the droplets because they will remain on a fixed location in the incubation chamber. In a further embodiment, the microfluidic device as disclosed herein provides that the microfluidic device further comprises at least one of:
a heating element for controlling the temperature of one or more incubation chambers;
a droplet mixer unit associated with each microfluidic channel for mixing each generated phage/bacteria droplet;
an inertial pump associated with each microfluidic channel, each inertial pump including a fluid actuator to pump fluid in the microfluidic channel;
a droplet selector unit associated with each microfluidic channel;
a UV illuminator;
an atmospheric control unit for controlling the atmospheric conditions during incubation;
a recirculation conduit for recirculating specific droplets to the input of the phage solution;
a phage selection/isolation unit for isolating and/or selecting a phage sample from a phage bank, an environmental sample.;
an upscaling unit for preparing a phage starter sample from a selected droplet;
an upscaling unit for preparing a bacterial starter sample from a selected droplet;
a phage solution titration unit;
a bacterial solution titration unit; and/or;.
anti bacterial solution titration unit;

As referred to herein an UV illuminator would allow continuous monitoring and selection of phage/bacteria droplets which are improved for lysis or non-lysis. The UV stress on the phages/bacteria results in an evolution towards a more potent phage by mutation or some non-lytic phages in bacteria emerge by UV stress of the bacterial population. Alternatively, the microfluidic chip may comprise a further fluid input for introducing mutagenic solutions into the droplets and obtaining similar results as with the UV illumination. The latter technique would include the steps of selecting a specific phage/bacteria droplet, adding the mutagenic or exposing the droplet to a mutagenic, breaking the droplets and adding a new dilution medium, fusing the droplets with new bacterial solution, starting over the incubation, selecting droplets with particular features and repeating the process thereby providing in an accelerated coevolution.

As referred to herein the atmospheric control unit may for instance be an air scrubber such that for instance for cyanobacteria oxygen production is guaranteed and the system does not fall in disarray during the incubation period.

In a further aspect, disclosed herein is a microfluidics-based platform comprising microfluidic device as disclosed herein and an analysis system for processing the data obtained from the monitoring system.

In a further aspect, disclosed herein is the use of a microfluidic device as disclosed herein for characterizing a phage solution comprises at least one of:
a quantitative assessment of the bacterial solution which includes determining the concentration of bacteria in the bacterial solution, preferably by determining the turbidity of the bacterial solution, the optical density of the bacterial solution and/or the colony forming units per ml (cfu/ml);
a qualitative assessment of the bacterial solution which includes the fitness, viability and/or identification of the bacterial solution;
a quantitative assessment of the phage solution which includes determining the concentration of viable phages in the phage solution, preferably by determining the plaque forming units per ml (pfu/ml); and/or
a qualitative assessment of the phage solution which includes the fitness and/or specificity of the phage solution.

The methods and devices as disclosed herein allows for the methods and devices to be used for a universal multitude of potential applications, allowing variations in phage/host combinations. Accordingly, the methods and devices as disclosed herein have applications in the field of phage therapy and various other possible fields of technology.

### EXAMPLES

### Example 1

The method as disclosed herein was executed as follows:

A dilution of purified, isolated phages (10⁶ pfu/ml) in a growth medium was prepared. A dilution of a bacterial strain of interest (10⁸ cfu/ml) in a growth medium was prepared. Both solutions were mixed in a 1:1 ratio. The phage/bacteria solution is introduced in a microfluidic device as disclosed herein in a liquid flow with oil and detergent in a Y droplet generator to generate droplets of 100-1000 pl (=10⁻⁶-10⁻⁷ ml, diameter 57-124 μm containing a culturable solution of bacteria 5 cfu/100 pl to 50 cfu/1000 pl with or without a concentration range of 0,005 to 5 pfu/100 pl to/1000 pl phage per droplet). Depending on the experiment approximately between 1000 to 25000 droplets are generated and brought to the transparent incubation chamber. The incubation occurs at a defined temperature (20 to 40° C.), atmosphere (anaerobic=N₂, Ar, aerobic=0₂, other atmospheric compositions may apply), time (2 h to 24 h) and luminance, depending on the type of bacteria, the motility and the growth rate.

Prior to the analysis an image without droplets is made from the incubation chamber. At time 0 a first image is taken from the incubation chamber and images are further taken as a timelapse during the entire incubation period, finishing with a final image at the end of the incubation period.

Images taken during the experiment are transferred to a computer analysis unit and further processed. The further processing for instance includes the rejection of features which are considered either too small or too large, or features with a non-uniform shape (not round, dust particles).

The remaining features are further analyzed.

The lysis in the droplets is determined by identifying the control droplets with only bacteria and identifying samples holding droplets with only bacteria and droplets where lysis occurred and quantification of the phages in the droplets.

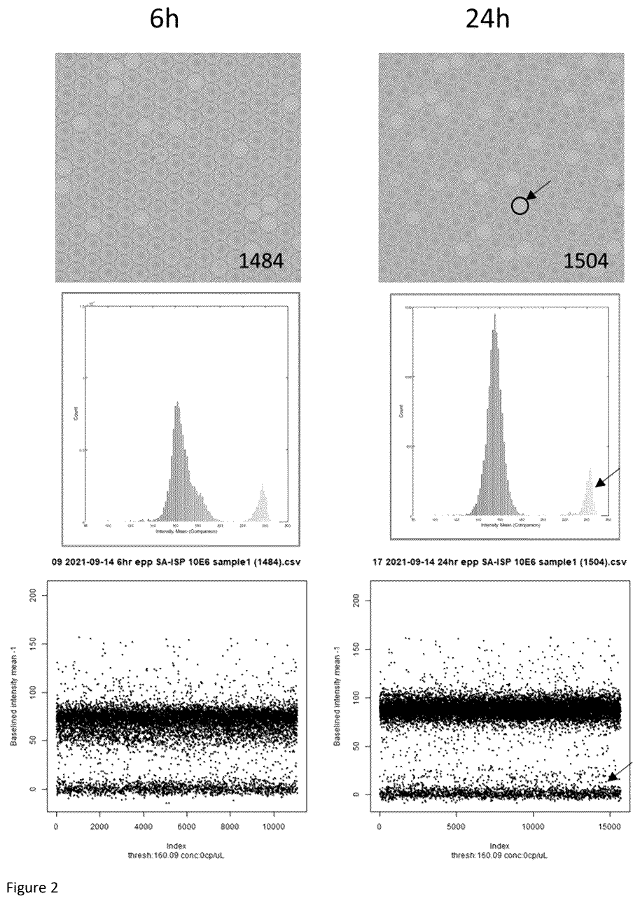

FIG. 2 shows an example of an experiment where an example bacteria: S aureus is challenged with ISP phage. Until 2 h computer analysis observes only one type of droplets. At 4 h and thereafter two distinct populations of droplets emerge representing the droplets with only bacteria on the one hand and droplets where phages have destroyed the bacterial population on the other hand. Both populations are clearly separated from one another. These populations can be accurately quantified with Poisson statistics to determine the viable concentration of phages.

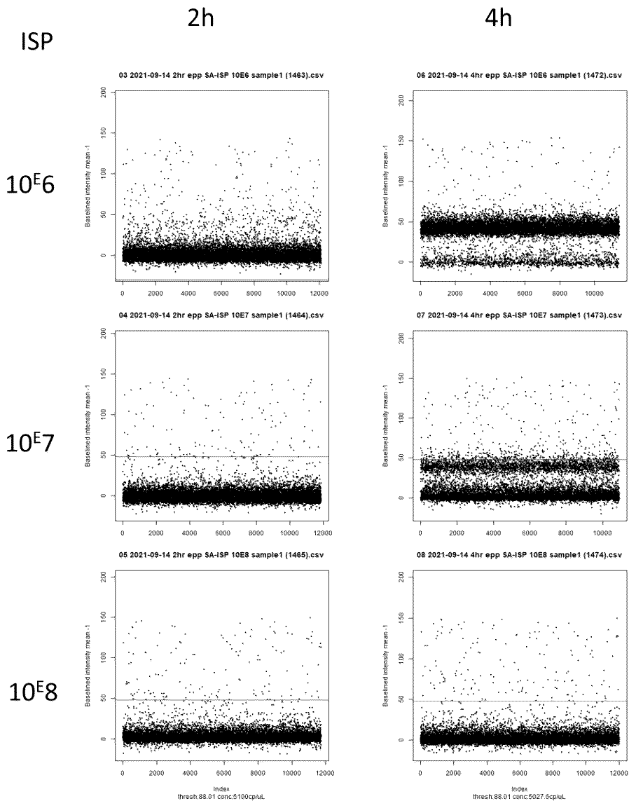

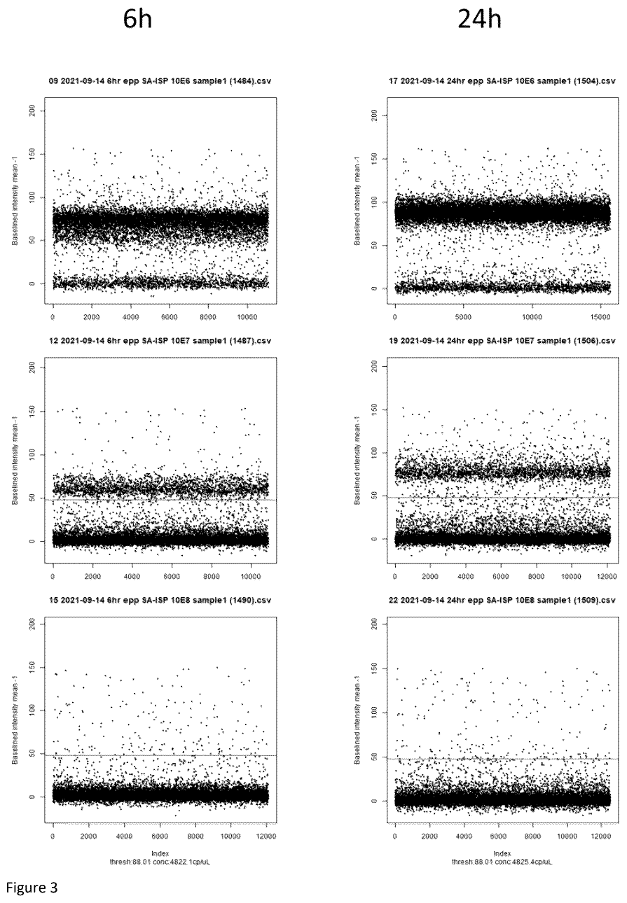

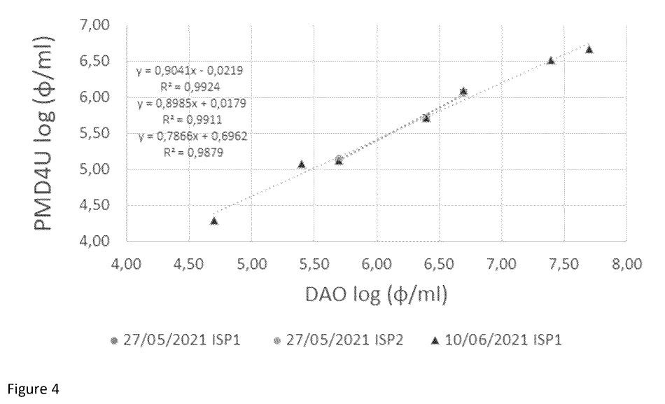

FIG. 3 shows the effect of overloading the experiment with phages until there is one or more than one phage in every droplet as demonstrated with the concentration range of 10⁸ PFU/ml phages. At this high concentration there are no droplets left wherein bacteria are able to grow. FIG. 4 shows the reproducibility of the method in several independent experiments at different dilutions as compared to the DAO method.

### Example2

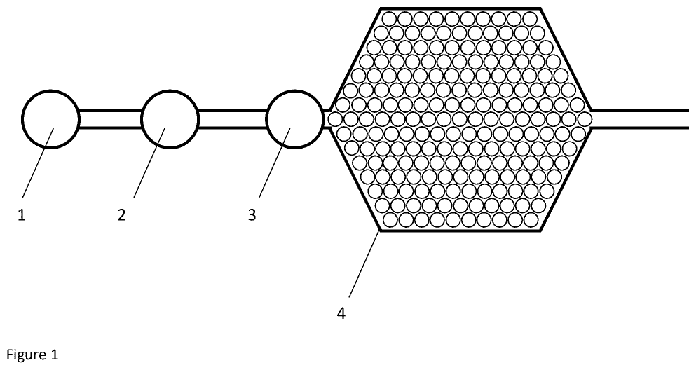

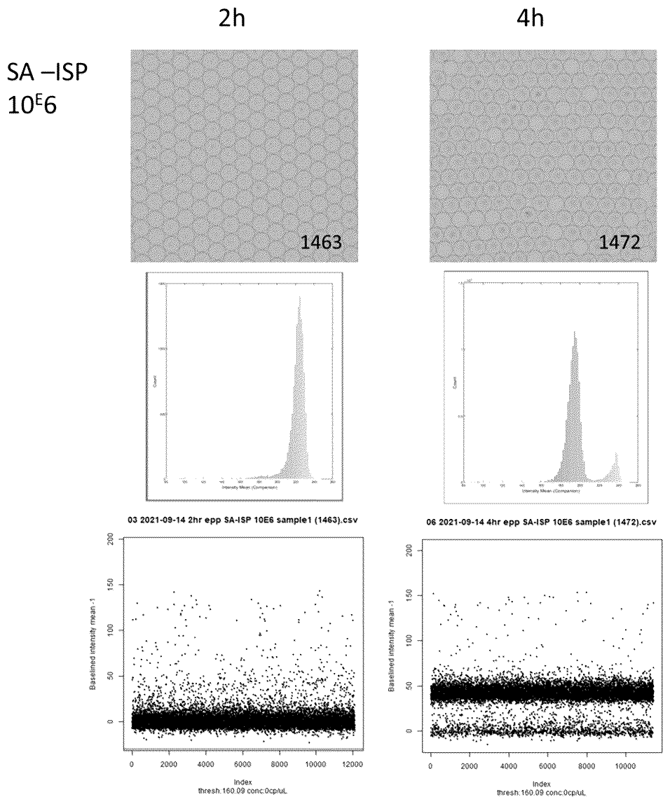

A device as disclosed herein is shown in FIG. 1 comprising a first fluid inlet for an oil solution (1), a second fluid input for a mixed phage—bacterial fluid input (2), a droplet generator unit (3) and a transparent incubation chamber (4) where the generated phage/bacteria droplets are incubated and monitored by the monitoring system.

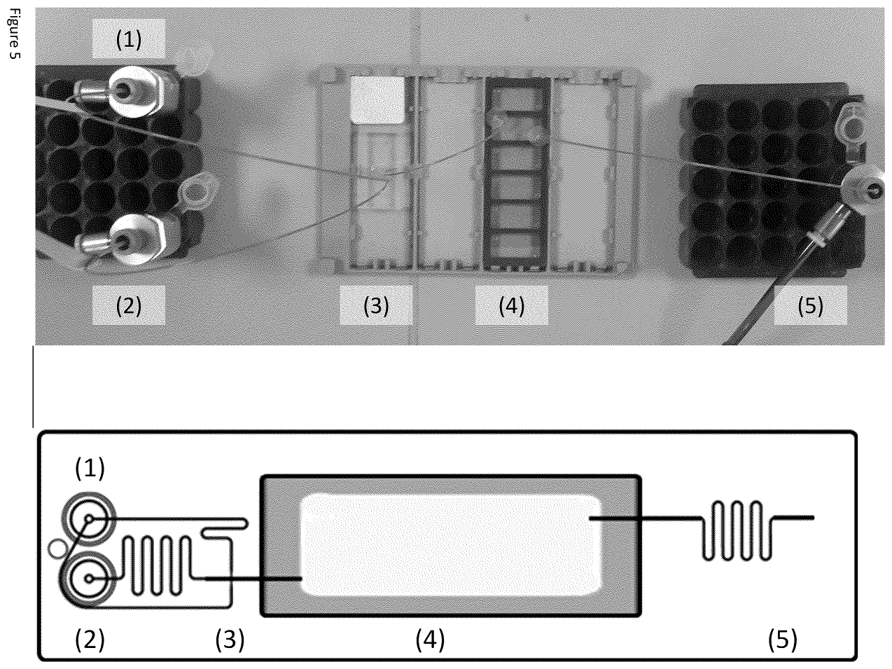

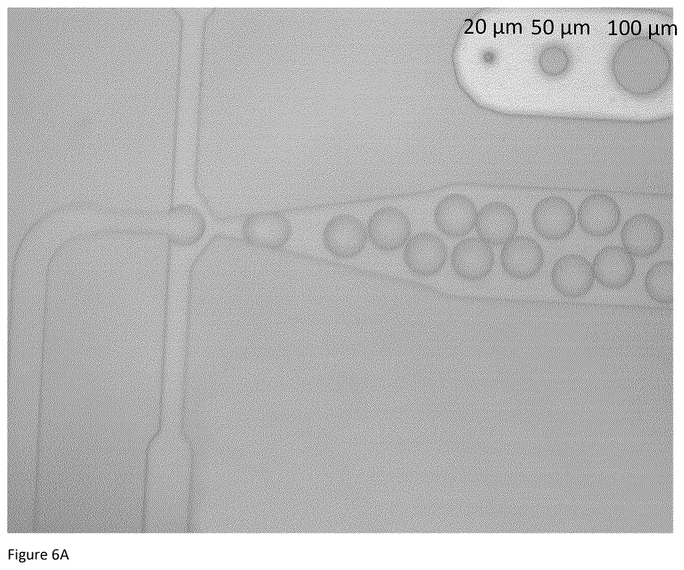

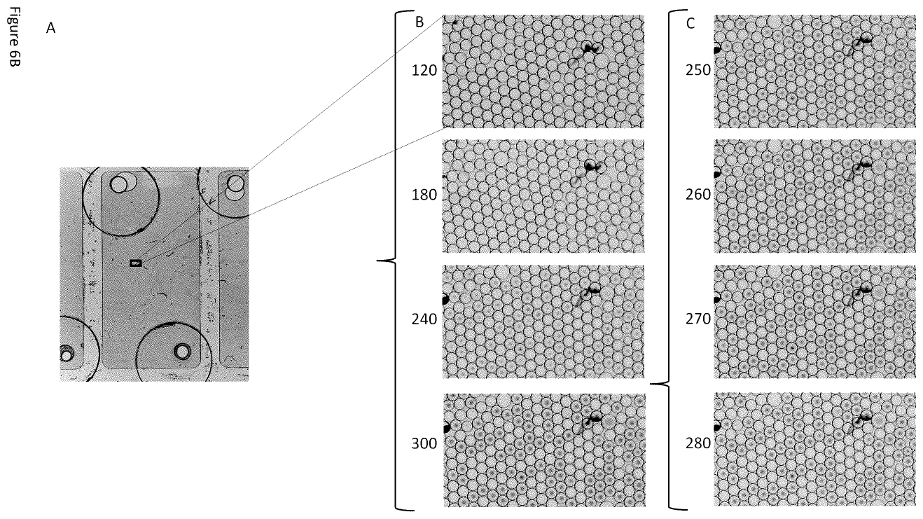

Also FIG. 5 shows a device as disclosed herein comprising a mixed phage-bacterial fluid input (2) and a second fluid input for an oil solution (1), a droplet generator unit (3) and a transparent incubation chamber (4). The outlet of the incubation chamber (4) is connected to collection means (5). FIG. 6A and 6B respectively show a detail of the droplet generator unit (3) and the incubation chamber (4). In FIG. 6B stills are shown of a timelapse experiment where an incubation chamber is incubated at 37° C. and illuminated for 500 ms each 10 min using darkfield. Panel A shows the incubation chamber which was loaded from the droplet generator. Images were taken at 10 minute time intervals. Panel B and C show a small zoomed out part of one incubation chamber holding Staphylococcus Aureus and ISP phage. Panel B shows some stills at intervals of 60 min. Panel C shows stills at intervals of 10 min in a timeframe between 240 minutes and 300 minutes. Growth and lysis are monitored continuously on chip and appear more clearly with time.

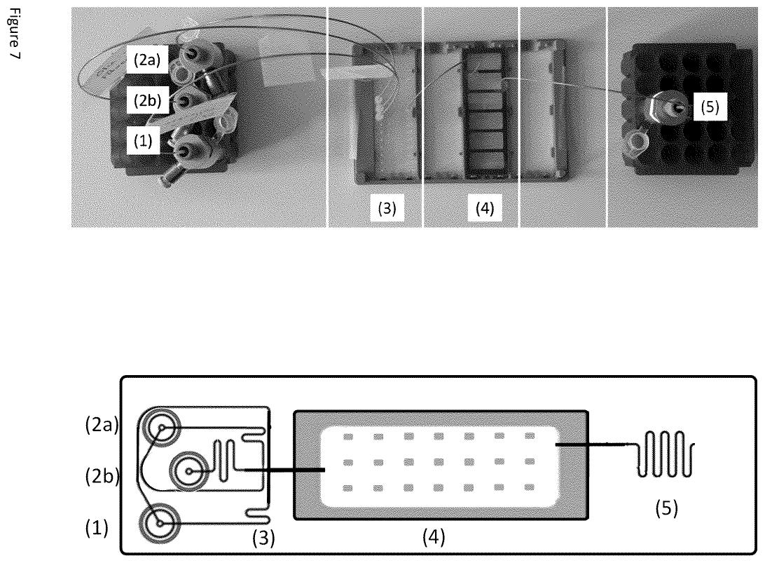

FIG. 7 shows a device similar to the one shown in FIG. 5, with the exception that the device further comprises a mixer unit for mixing the phage solution (2a) or a dilution thereof with a bacterial solution (2b), thereby generating a phage/bacteria solution.

What is claimed is:

1-15. (canceled)

16. A method for characterizing a phage solution in a microfluidic device comprising the steps of:
mixing a phage solution or a dilution thereof with a bacterial solution, thereby generating a phage/bacteria solution; and through a set of microfluidic channels of said microfluidics device:
i. pumping the phage/bacteria solution to a droplet generator unit, thereby generating phage/bacteria droplets;
ii. incubating the generated phage/bacteria droplets in a transparent incubation chamber; and;
iii. monitoring, in function of time, the characteristics of the phage/bacteria droplets in said incubation chamber.

17. The method according to claim 16, wherein the monitoring of the characteristics of the phage/bacteria droplets comprises at least one of:
i. a quantitative assessment of the bacterial solution which includes determining the concentration of bacteria in the bacterial solution, optionally wherein the determining the turbidity of the bacterial solution, the optical density of the bacterial solution and/or the colony forming units per ml (cfu/ml);
ii. a qualitative assessment of the bacterial solution which includes assessment of fitness, viability and/or identification of the bacterial solution;
iii. a quantitative assessment of the phage solution which includes determining the concentration of viable phages in the phage solution, optionally wherein the determining the plaque forming units per ml (pfu/ml); and
iv. a qualitative assessment of the phage solution which includes the fitness and/or specificity of the phage solution.

18. The method according to claim 16, wherein the step of mixing a phage solution or a dilution thereof with a bacterial solution occurs on the microfluidic device.

19. The method according to claim 16, wherein the incubation step occurs under predetermined conditions which includes a predetermined incubation temperature, a predetermined atmosphere and a predetermined incubation time.

20. The method according to claim 16, wherein the step of monitoring, in function of time, the characteristics of the phage/bacteria droplets in said incubation chamber includes obtaining a control image at the start of the incubation period and obtaining a final image at the end of the incubation period, and optionally obtaining images at preset intervals during the incubation period.

21. The method according to claim 16, wherein between 1000 and 25000 phage/bacteria droplets are simultaneously monitored in said transparent incubation chamber.

22. The method according to claim 16, wherein the method further comprises the step of guiding incubated phage/bacteria droplets to a droplet selector and collecting selected phage/bacteria droplets.

23. A microfluidic phage solution characterization device, comprising:
i. a fluid input;
ii. a fluid output; and
iii. one or more microfluidic channels fluidically connecting the fluid input to the fluid output wherein each microfluidic channel comprises a droplet generator unit for generating phage/bacteria droplets, a transparent incubation chamber for incubating the generated phage/bacteria droplets and a monitoring system for, in function of time, determining the characteristics of phage/bacteria droplets in said incubation chamber.

24. The microfluidic phage solution characterization device according to claim 23, further comprising:
i. a first fluid input for a phage solution;
ii. a second fluid input for a bacterial solution;
iii. a third fluid input for an oily solution; and optionally
iv. a fourth fluid input for a titration solution;
and wherein each microfluidic channel further comprises a mixer unit for mixing the phage solution or a dilution thereof with a bacterial solution, thereby generating a phage/bacteria solution.

25. The microfluidic phage solution characterization device according to claim 23, wherein the droplet generator unit is a passive or active droplet generator unit.

26. The microfluidic phage solution characterization device according to claim 23, wherein the transparent incubation chamber has a height between 50 μm and 500 μm.

27. The microfluidic phage solution characterization device according to claim 23, wherein the transparent incubation chamber has a flat or waved surface.

28. The microfluidic phage solution characterization device according to claim 23, wherein the microfluidic phage solution characterization device further comprises at least one of:
i. a heating element for controlling the temperature of one or more incubation chambers;
ii. a droplet mixer unit associated with each microfluidic channel for mixing each generated phage/bacteria droplet;
iii. an inertial pump associated with each microfluidic channel, each inertial pump including a fluid actuator to pump fluid in the microfluidic channel;
iv. a droplet selector unit associated with each microfluidic channel;
v. a UV illuminator;
vi. an atmospheric control unit for controlling the atmospheric conditions during incubation;
vii. a recirculation conduit for recirculating specific droplets to the input of the phage solution;
viii. a phage selection/isolation unit for isolating and/or selecting a phage sample from a phage bank, an environmental sample;
ix. an upscaling unit for preparing a phage starter sample from a selected droplet;
x. an upscaling unit for preparing a bacterial starter sample from a selected droplet;
xi. a phage solution titration unit; and
xii. a bacterial solution titration unit.

29. A microfluidics-based platform comprising the microfluidic device according to claim 15 and an analysis system for processing the data obtained from the monitoring system.

30. A microfluidic device according to claim 15, wherein the microfluidic device characterizes a phage solution comprising at least one of:
i. a quantitative assessment of the phage solution which includes determining the concentration of viable phages in the phage solution, preferably by determining the plaque forming units per ml (pfu/ml); and/or
ii. a qualitative assessment of the phage solution which includes the fitness and/or specificity of the phage solution.
